(19) United States
(12) Patent Application Publication
(10) Pub. No.: US 2020/0407771 A1
(43) Pub. Date: Dec. 31, 2020

(54) THRESHOLD-STIMULATED PLASMA KALLIKREIN ACTIVITY AS A BIOMARKER FOR DIAGNOSIS OF BRADYKININ-MEDIATED ANGIOEDEMA

(71) Applicant: The Regents of the University of California, Oakland, CA (US)

(72) Inventors: Bruce Zuraw, San Diego, CA (US); Maria Luz Lara-Marquez, San Diego, CA (US)

(21) Appl. No.: 16/971,664

(22) PCT Filed: Feb. 22, 2019
(86) PCT No.: PCT/US2019/019296

§ 371 Date: Aug. 20, 2020

Related U.S. Application Data
(60) Provisional application No. 62/633,604, filed on Feb. 22, 2018.

(51) Int. Cl. C12Q 1/37; C12Q 1/02

(52) U.S. Cl. CPC C12Q 1/37; C12Q 2600/112; C12Q 1/02

(57) ABSTRACT

Provided is a diagnostic test for the positive identification of patients with bradykinin-mediated angioedema. The test is valuable for distinguishing bradykinin-mediated angioedema from non-bradykinin-mediated angioedema. The test allows clinicians to clearly separate patients with bradykinin-mediated from histamine-mediated angioedema. Results can be obtained in under an hour, allowing for the proper treatment of angioedema based on the underlying etiology.

### CROSS-REFERENCE TO RELATED APPLICATIONS

This application claims priority to and benefit of U.S. Ser. No. 62/633,604, filed on Feb. 22, 2018, which is incorporated herein by reference in its entirety for all purposes.

### BACKGROUND

Recurrent angioedema without urticaria can be mediated by different mediators. predominantly histamine or bradykinin. Compared to histamine-mediated angioedema, bradykinin-mediated angioedema is distinguished by its increased severity, longer duration of swelling, and lack of response to treatment with antihistamines, corticosteroids or epinephrine (1). Differentiating bradykinin-from histamine-mediated angioedema is of critical importance to prevent morbidity and mortality (2). The ability to diagnose bradykinin-mediated angioedema with normal C1 inhibitor (C1INH), however, has been severely limited by the lack of any available diagnostic tests.

Hereditary angioedema (HAE) due to C1INH deficiency (HAE-C1INH) and acquired C1INH deficiency (ACID) are the best characterized forms of bradykinin-mediated angioedema (3-6). A novel form of HAE with normal C1INH (HAE-nl-C1INH) was initially described in 2000 (7,8). Subsequently a minority of HAE-nl-C1INH patients were found to have mutations in F12 (the gene encoding FXII; HAE-FXII) that segregated with disease activity (9-12). HAE-FXII is autosomal dominant with low penetrance (13). Most recently, mutations in angiopoietin-1 and plasminogen have also been linked to HAE-nl-C1INH (14,15). Most HAE-nl-C1INH patients, however, do not have a known mutation, and are called HAE-unknown. Distinguishing HAE-unknown from idiopathic histaminergic angioedema (IHA) is very challenging due to the lack of any definitive biomarker for either condition. Currently the diagnosis of HAE-unknown is based on consensus clinical criteria, though the sensitivity and specificity of this approach is unknown (16). Another group of angioedema patients is clinically similar to HAE-unknown, but lack a compelling family history of angioedema. This group has been classified as idiopathic non-histaminergic angioedema (INHA) and also lacks any biomarker to establish the diagnosis (17).

Recent evidence strongly supports the hypothesis that HAE-FXII is mediated by bradykinin (18,19). Circumstantial evidence suggests that HAE-unknown and INHA are also bradykinin-mediated (20,21), and may respond to bradykinin-targeted medications (13,22-24).

The lack of specific biomarkers makes the diagnosis of hereditary angioedema (HAE) with normal levels of C1-inhibitor (C1INH) protein (HAE-nl-C1INH) and idiopathic non-histaminergic angioedema (INHA) difficult. Confirming or excluding these diagnoses is a significant challenge for clinicians evaluating patients with angioedema.

### SUMMARY

In one aspect, provided are methods of identifying a patient suffering bradykinin-mediated angioedema. In some embodiments, the methods comprise:

a) subjecting or exposing a plasma sample from the patient to a kallikrein stimulating agent or an activator of the plasma contact system in an amount sufficient to stimulate kallikrein protease activity;

b) measuring the kallikrein protease activity; and

c) identifying an increased level of kallikrein protease activity in comparison to kallikrein protease activity from a plasma sample of a normal control as indicative that the patient is suffering bradykinin-mediated angioedema. In some embodiments, the methods further comprise administering a bradykinin-targeted medication to the patient. In some embodiments, the patient is administered an agent that inhibits the release of bradykinin (e.g., a plasma-derived C1-inhibitor, a recombinant C1-inhibitor, a kallikrein inhibitor) and/or an agent that prevents bradykinin from binding to its receptor (e.g., a bradykinin B2 receptor antagonist). In some embodiments, the method identifies patients suffering hereditary angioedema due to C1 inhibitor deficiency (HAE-C1INH), hereditary angioedema with normal C1 inhibitor (HAE-nl-C1INH) and/or idiopathic non-histaminergic angioedema (INHA).

In another aspect, provided are methods of identifying a patient suffering histamine-mediated angioedema. In some embodiments, the methods comprise:

a) subjecting or exposing a plasma sample from the patient to a kallikrein stimulating agent or an activator of the plasma contact system in an amount sufficient to stimulate kallikrein protease activity;

b) measuring the kallikrein protease activity; and

c) identifying an essentially equivalent level of kallikrein protease activity in comparison to kallikrein protease activity from a plasma sample of a normal control as indicative that the patient is suffering histamine-mediated angioedema. In some embodiments, the methods further comprise administering a histamine-targeted medication, e.g., Cetirizine, (Highly specific Histamine receptor Type 1 antagonist), to the patient.

In some embodiments of the methods, the kallikrein stimulating agent or the activator of the plasma contact system comprises dextran sulfate (DXS). In some embodiments, the plasma sample is subjected or exposed to kallikrein stimulating agent or the activator of the plasma contact system at one or more concentrations in the range of from about 1 μg/mL to about 10 μg/mL, e.g., 1 μg/mL, 1.25 μg/mL, 2 μg/mL, 2.5 μg/mL, 3 μg/mL, 4 μg/mL, 5 μg/mL, 6 μg/mL, 7 μg/mL, 8 μg/mL, 9 μg/mL, or 10 μg/mL. In some embodiments, the plasma subjected or exposed to the kallikrein stimulating agent or the activator of the plasma contact system is contacted with a kallikrein substrate. In some embodiments, the kallikrein substrate comprises Z-Phe-Arg-AMC-HCl. In some embodiments, cleavage of the kallikrein substrate is detected via a fluorescent, chromogenic, chemiluminescent, radioactive or enzymatic signal. In some embodiments, the plasma sample is subjected or exposed to kallikrein stimulating agent or the activator of the plasma contact system for 60 minutes or less, e.g., 50 minutes, 40 minutes, 30 minutes, or less.

Additionally or alternatively, various embodiments contemplated herein may include, but need not be limited to, one or more of the following

Embodiment 1: A method of distinguishing a patient suffering bradykinin-mediated angioedema from a patient suffering from histamine-mediated angioedema, comprising:
stimulating a plasma sample from the patient with a threshold amount of a kallikrein stimulating agent or an activator of the plasma contact system;
measuring kallikrein protease activity in said sample; and
identifying an increased level of kallikrein protease activity in comparison to kallikrein protease activity from a plasma sample of a normal control as indicative that the patient is suffering bradykinin-mediated angioedema; or
identifying an essentially equivalent level of kallikrein protease activity in comparison to kallikrein protease activity from a plasma sample of a normal control as indicative that the patient is suffering histamine-mediated angioedema.

Embodiment 2: The method of embodiment 1, wherein said patient is a patient presenting with one or more symptoms of angioedema.

Embodiment 3: The method of embodiment 2, wherein said symptoms of angioedema comprise one or more symptoms selected from: swelling of one or more of the hands, feet, area around the eyes, lips, tongue, genitals, and/or inside of the throat; and/or difficulty breathing; and/or dizziness or fainting.

Embodiment 4: The method according to any one of embodiments 1-3, wherein said increased level of kallikrein protease activity in comparison to kallikrein protease activity from a plasma sample of a normal control comprises a statistically significant increased level at a 5%, or at a 1% confidence level.

Embodiment 5: The method according to any one of embodiments 1-4, wherein said increased level of kallikrein protease activity comprises a kallikrein activity level at least 50%, or at least 50%, or at least 60%, or at least 70%, or at least 80%, or at least 90%, or at least 1.5 fold, or at least 2 fold, or at least 3 fold, or at least 4-fold, or at least 5-fold higher than said normal control.

Embodiment 6: The method according to any one of embodiments 1-4, wherein said increased level of kallikrein protease activity comprises a kallikrein activity level at least 5-fold higher than said normal control.

Embodiment 7: The method according to any one of embodiments 1-6, wherein said substantially equivalent level of kallikrein protease activity in comparison to kallikrein protease activity from a plasma sample of a normal control comprises a statistically insignificant difference from said control at a 5%, or at a 1% confidence level.

Embodiment 8: The method according to any one of embodiments 1-7, wherein said essentially equivalent level of kallikrein protease activity comprises a kallikrein protease activity level within ±20%, or within ±15%, or within ±10%, or within ±5% of said control level.

Embodiment 9: The method of embodiment 8, wherein said essentially equivalent level of kallikrein protease activity comprises a kallikrein protease activity level within ±20% of said control level.

Embodiment 10: The method according to any one of embodiments 1-9, wherein said normal control comprises a level of kallikrein protease activity determined from a population known not to have angioedema.

Embodiment 11: The method according to any one of embodiments 1-9, wherein said normal control comprises a level of kallikrein protease activity from a subject determined not to have angioedema.

Embodiment 12: The method of embodiment 11, wherein said level of kallikrein protease activity from a subject determined not to have angioedema comprises a level of kallikrein protease activity determined from said patient at a time when said subject patient is asymptomatic for angioedema.

Embodiment 13: The method of embodiment 11, wherein said level of kallikrein protease activity from a subject determined not to have angioedema comprises a level of kallikrein protease activity determined from a subject that is not said patient.

Embodiment 14: The method according to any one of embodiments 1-13, wherein the kallikrein stimulating agent or the activator of the plasma contact system comprises an agent selected from the group consisting of dextran sulfate, or polyphosphates.

Embodiment 15: The method of embodiment 14, wherein the kallikrein stimulating agent or the activator of the plasma contact system comprises dextran sulfate (DXS).

Embodiment 16: The method of any one of embodiments 1-15, wherein the plasma sample is subjected to or exposed to kallikrein stimulating agent or the activator of the plasma contact system at one or more concentrations in the range of from about 0.5 μg/mL, or from about 1.0 μg/mL, or from about 1.25 μg/mL up to about 10 μg/mL, or up to about 9 μg/mL, or up to about 8 μg/mL, or up to about 7 μg/mL, or up to about 6 μg/mL, or up to about 5 μg/mL, or up to about 4 μg/mL, or up to about 3 μg/mL, or up to about 2 μg/mL.

Embodiment 17: The method of embodiment 16, wherein the plasma sample is subjected to or exposed to kallikrein stimulating agent or the activator of the plasma contact system a concentration ranging from about 1 μg/mL up to about 5 μg/mL, or from about 1.25 μg/mL up to about 4 μg/mL, or at from about 1.5 μg/mL up to about 3 μg/mL, or at about 2.0 μg/mL up to about 3.0 μg/mL.

Embodiment 18: The method of any one of embodiments 1-17, wherein measuring kallikrein protease activity in said sample comprises stimulating said sample with a kallikrein substrate and detecting and/or quantifying cleavage of said substrate.

Embodiment 19: The method of embodiment 18, wherein the kallikrein substrate comprises Z-Phe-Arg-AMC-HCl.

Embodiment 20: The method according to any one of embodiments 18-19, wherein cleavage of the kallikrein substrate is detected via a fluorescent, chromogenic, chemiluminescent, radioactive or enzymatic signal.

Embodiment 21: The method of any one of embodiments 1-20, wherein the plasma sample is subjected or exposed to kallikrein stimulating agent or the activator of the plasma contact system for about 60 minutes or less, or for about 50 minutes or less, or for about 40 minutes or less, or for about 30 minutes or less. or for about 20 minutes or less, or for about 10 minutes or less.

Embodiment 22: The method according to any one of embodiments 1-21, wherein the kallikrein protease activity level(s) are normalized to rKLKB1.

Embodiment 23: The method according to any one of embodiments 1-22, wherein said plasma sample comprises a frozen sample that has been thawed once that has been thawed once at 37° C. to be assayed.

Embodiment 24: The method according to any one of embodiments 1-22, wherein said plasma sample comprises a fresh (unfrozen) plasma sample.

Embodiment 25: The method according to any one of embodiments 1-22, wherein said plasma sample has been stored as a frozen plasma sample.

Embodiment 26: The method according to any one of embodiments 1-25, wherein said identifying an identifying an increased level of kallikrein protease activity comprises diagnosing said patient as a patient with a bradykinin-mediated angioedema.

Embodiment 27: The method of embodiment 26, where said diagnosing is in the context of a differential diagnosis for bradykinin-mediated angioedema.

Embodiment 28: The method according to any one of embodiments 26-27, wherein said diagnosing comprises identifying said patient as a patient suffering hereditary angioedema due to C1 inhibitor deficiency (HAE-C1INH), hereditary angioedema with normal C1 inhibitor (HAE-nl-CINH) and/or idiopathic non-histaminergic angioedema (INHA).

Embodiment 29: The method according to any one of embodiments 1-25, wherein said identifying an identifying an essentially equivalent level of kallikrein protease activity comprises diagnosing said patient as a patient with a histamine-mediated angioedema.

Embodiment 30: The method of embodiment 29, where said diagnosing is in the context of a differential diagnosis for histamine-mediated angioedema.

Embodiment 31: The method according to any one of embodiments 1-30, wherein said method further comprises recording said level of kallikrein protease activity in a patient medical record.

Embodiment 32: The method according to any one of embodiments 1-31, wherein said method further comprises recording said angioedema as a bradykinin-mediated angioedema or a histamine-mediated angioedema in a medical record.

Embodiment 33: The method according to any one of embodiments 1-31, wherein said method further comprises recording in a medical record said subject as a subject with hereditary angioedema due to C1 inhibitor deficiency (HAE-C1INH), hereditary angioedema with normal C1 inhibitor (HAE-nl-CINH) and/or idiopathic non-histaminergic angioedema (INHA).

Embodiment 34: The method according to any one of embodiments 31-33, wherein the patient medical record is a medical record maintained by a laboratory, physician's office, a hospital, a health maintenance organization, an insurance company, or a personal medical record website.

Embodiment 35: The method according to any one of embodiments 31-34, wherein information from said medical record is recorded on or in a medic alert article selected from a card, worn article, or radiofrequency identification (RFID) tag.

Embodiment 36: A method of treating a patient for angioedema, said method comprising: using a plasma sample from said patient, performing a method according to any one of embodiments 1-35 to identify said subject as having a bradykinin-mediated angioedema or a histamine-mediated angioedema; and where said subject is identified as having a bradykinin-mediated angioedema, treating said subject for bradykinin-mediated angioedema; and where said subject is identified as having a histamine-mediated angioedema, treating said subject for histamine-mediated angioedema.

Embodiment 37: A method of treating a subject for angioedema, said method comprising: in a subject identified by a method according to any one of embodiments 1-35 as having a bradykinin-mediated angioedema, treating said subject for bradykinin-mediated angioedema; or in a subject identified by a method according to any one of embodiments 1-35 as having a histamine-mediated angioedema, treating said subject for histamine-mediated angioedema.

Embodiment 38: The method according to any one of embodiments 36-37, wherein said treating said subject for bradykinin-mediated angioedema comprises administering to, or causing to be administered to, said subject a bradykinin-targeted medication.

Embodiment 39: The method of embodiment 38, wherein said bradykinin-targeted medication comprises one or more agents selected from the group consisting of IV fresh frozen plasma (FFP), epsilon-aminocaproic acid, C1-INH replacement therapy, a plasma kallikrein inhibitor, a C1 esterase inhibitor, and a bradykinin receptor blocker.

Embodiment 40: The method of embodiment 39, wherein said bradykinin-targeted medication comprises C1-INH (Berinert).

Embodiment 41: The method according to any one of embodiments 39-40, wherein said bradykinin-targeted medication comprises the plasma kallikrein inhibitor ecallantide or ladalenumab (Takhzyro®) (a humanized monoclonal antibody against Kallikrein).

Embodiment 42: The method according to any one of embodiments 39-41, wherein said bradykinin-targeted medication comprises the bradykinin receptor blocker icatiban.

Embodiment 43: The method according to any one of embodiments 39-42, wherein said bradykinin-targeted medication comprises the C1 esterase inhibitor, plasma sources (Berinert,® Haegarda®, Cynrize® or recombinant ruconest.

Embodiment 44: The method according to any one of embodiments 36-37, wherein said treating said subject for histamine-mediated angioedema comprises administering to, or causing to be administered to, said subject a histamine-targeted medication.

Embodiment 45: The method of embodiment 38, wherein said histamine targeted medication comprises one or more agents selected from the group consisting of H1 receptor antagonists and drugs that are not histamine targeted but that are needed due to the adrenergic pathways and inflammatory response that become activated by histamine release (e.g., epinephrine, an antihistamine, a β2 agonist, a corticosteroid.

Embodiment 46: The method of embodiment 45, wherein said histamine targeted medication comprises epinephrine.

Embodiment 47: The method according to any one of embodiments 45-46, wherein said histamine targeted medication comprises a corticosteroid.

Embodiment 48: The method of embodiment 47, wherein said corticosteroid comprises methylprednisolone.

Embodiment 49: The method according to any one of embodiments 45-48, wherein said histamine targeted medication comprises an antihistamine.

Embodiment 50: The method according to any one of embodiments 45-49, wherein said B2 receptors targeted medication comprises a β2 agonist.

Embodiment 51: The method of embodiment 50, wherein said β2 agonist comprises albuterol.

Embodiment 52: The method according to any one of embodiments 45-51, wherein said histamine-targeted medication comprises a mast-cell targeted medication comprising a leukotriene modifier or omalizumab.

Embodiment 53: A method of identifying a patient suffering bradykinin-mediated angioedema, comprising:
a) subjecting or exposing a plasma sample from the patient to a kallikrein stimulating agent or an activator of the plasma contact system in an amount sufficient to stimulate kallikrein protease activity;
b) measuring the kallikrein protease activity; and
c) identifying an increased level of kallikrein protease activity in comparison to kallikrein protease activity from a plasma sample of a normal control as indicative that the patient is suffering bradykinin-mediated angioedema.

Embodiment 54: The method of embodiment 53, further comprising administering a bradykinin-targeted medication to the patient.

Embodiment 55: The method of any one of embodiments 53 to 54, wherein the kallikrein stimulating agent or the activator of the plasma contact system comprises dextran sulfate (DXS).

Embodiment 56: The method of any one of embodiments 53 to 55, wherein the plasma sample is subjected or exposed to kallikrein stimulating agent or the activator of the plasma contact system at one or more concentrations in the range of from about 1 μg/mL to about 10 μg/mL, e.g., 1 μg/mL, 1.25 μg/mL, 2 μg/mL, 2.5 μg/mL 3 μg/mL, 4 μg/mL, 5 μg/mL, 6 μg/mL, 7 μg/mL, 8 μg/mL, 9 μg/mL, or 10 μg/mL.

Embodiment 57: The method of any one of embodiments 53 to 56, wherein the plasma subjected or exposed to the kallikrein stimulating agent or the activator of the plasma contact system is contacted with a kallikrein substrate.

Embodiment 58: The method of embodiment 57, wherein the kallikrein substrate comprises Z-Phe-Arg-AMC-HCl.

Embodiment 59: The method of embodiment 57, wherein cleavage of the kallikrein substrate is detected via a fluorescent, chromogenic, chemiluminescent, radioactive or enzymatic signal.

Embodiment 60: The method of any one of embodiments 53 to 59, wherein the plasma sample is subjected or exposed to kallikrein stimulating agent or the activator of the plasma contact system for 60 minutes or less, e.g., 50 minutes, 40 minutes, 30 minutes, or less.

Embodiment 61: The method of any one of embodiments 53 to 60, wherein the method identifies patients suffering hereditary angioedema due to C1 inhibitor deficiency (HAE-C1INH), hereditary angioedema with normal C1 inhibitor (HAE-nl-CINH) and/or idiopathic non-histaminergic angioedema (INHA).

Embodiment 62: A method of identifying a patient suffering histamine-mediated angioedema, comprising:
a) subjecting or exposing a plasma sample from the patient to a kallikrein stimulating agent or an activator of the plasma contact system in an amount sufficient to stimulate kallikrein protease activity;
b) measuring the kallikrein protease activity; and
c) identifying an essentially equivalent level of kallikrein protease activity in comparison to kallikrein protease activity from a plasma sample of a normal control as indicative that the patient is suffering histamine-mediated angioedema.

Embodiment 63: The method of embodiment 62, further comprising administering a histamine-targeted medication to the patient.

Embodiment 64: The method of any one of embodiments 62 to 63, wherein the kallikrein stimulating agent or the activator of the plasma contact system comprises dextran sulfate (DXS).

Embodiment 65: The method of any one of embodiments 62 to 64, wherein the plasma sample is subjected or exposed to kallikrein stimulating agent or the activator of the plasma contact system at one or more concentrations in the range of from about 1 μg/mL to about 10 μg/mL, e.g., 1 μg/mL, 1.25 μg/mL, 2 μg/mL, 2.5 μg/mL, 3 μg/mL, 4 μg/mL, 5 μg/mL, 6 μg/mL, 7 μg/mL, 8 μg/mL, 9 μg/mL, or 10 μg/mL.

Embodiment 66: The method of any one of embodiments 62 to 65, wherein the plasma subjected or exposed to the kallikrein stimulating agent or the activator of the plasma contact system is contacted with a kallikrein substrate.

Embodiment 67: The method of embodiment 66, wherein the kallikrein substrate comprises Z-Phe-Arg-AMC-HCl.

Embodiment 68: The method of embodiment 66, wherein cleavage of the kallikrein substrate is detected via a fluorescent, chromogenic, chemiluminescent, radioactive or enzymatic signal.

Embodiment 69: The method of any one of embodiments 62 to 68, wherein the plasma sample is subjected or exposed to kallikrein stimulating agent or the activator of the plasma contact system for 60 minutes or less, e.g., 50 minutes, 40 minutes, 30 minutes, or less.

### Definitions

The term “hereditary angioedema” (“HAE”) as used herein relates to angioedema caused by a low content and low inhibitory activity of C1-INH in the circulation (HAE type I) or by the presence of normal or elevated antigenic levels of C1-INH of low functional activity (HAE type II). The term “HAE” as used herein also encompasses HAE with normal C1-INH (HAE-nl-C1INH) which has been described recently in two subcategories: (1) HAE due to mutation in the factor XII gene and, as a result, increased activity of factor XII leading to a high generation of bradykinin, (also known as HAE Type III) and (2) HAE of unknown genetic cause (HAE-U). Patients characterized as HAE, all have documented family history. A subset of patients may have the same clinical phenotype, non-responsive to anti-histamines, that are known as Idiopathic Non-histaminergic-Angioedema (INHA) but do not have family history. Patients suffering from hereditary angioedema or INHA, have edema attacks that can occur in various intervals, including a daily, weekly, monthly, or even yearly basis. Furthermore, there are affected patients wherein no edema occurs.

The term “angioedema” (“edema”) as used herein relates to swelling of tissue, for example swelling of skin or mucosa. The swelling can occur, for example, in the face, at hands or feet or on the genitals. Furthermore, swelling can occur in the gastro-intestinal tract or in the respiratory tract. Other organs can also be affected. Swelling persists usually between one and three days. However, remission can already occur after hours or not until weeks.

A “maximal amount” or “optimal dose” of a kallikrein stimulating agent or an activator of the plasma contact system refers to an amount/dose of stimulating agent or activator that elicits a measurable Kallikrein activity in all subjects independent whether the subjects have any swelling disorder, normal non-swelling controls, and all histaminergic and bradykinin mediated angioedema patients without any significant differences between the means of the Kallikrein activity measured.

A “threshold amount” of a kallikrein stimulating agent or an activator of the plasma contact system refers to an amount/dose of stimulating agent or activator that elicits a minimal amount of kallikrein protease activity in normal plasma but elicits a significant amount of kallikrein activity protease activity in plasma from in subjects with bradykinin-mediated angioedema. This dose may vary depending on the specific activator or stimulator used, but can readily be defined more using recombinant plasma kallikrein as a standard. For a threshold amount of any given stimulating agent or activator, plasma from bradykinin-mediated angioedema subjects will show a significant (e.g., statistically significant at a 5%, or at a 1%, or at a 0.1% confidence level) mean kallikrein protease activity level above the mean kallikrein protease activity level from normal plasma. In certain embodiments the threshold amount of a stimulating agent or activator will produce a mean kallikrein protease activity level in plasma from subjects with a bradykinin-mediated angioedema at least 2 SD above the mean kallikrein protease activity level from normal plasma.

### BRIEF DESCRIPTION OF THE DRAWINGS

FIG. 1, panels A-F, illustrates that dextran sulfate (DXS) stimulates kallikrein activity in a dose-dependent manner. Plasma was stimulated ex vivo with various doses of DXS as indicated, and plasma kallikrein activity measured as RFU over 30 minutes. BG refers to background or spontaneous kallikrein activity. Panels are as follows: A: non-swelling control; Panel B: histaminergic angioedema; Panel C: HAE-C1INH; D, HAE-FXII; Panel E: HAE-U; and Panel F: INHA.

FIG. 2, panels A-B. Stimulated plasma kallikrein activity assay is specific for contact system activation. Panel A: Normal plasma was stimulated with DXS 5 μg/ml in the absence (red line) or presence (blue line) of CTI. Spontaneous kallikrein activity is shown by the black line. Panel B: Spontaneous kallikrein activity in normal plasma is shown by the black line. Kallikrein activity in plasma kallikrein deficient plasma stimulated with DXS 5 μg/ml is shown by the red line.

FIG. 3, panels A-B. Kallikrein activity is increased in subjects in the HAE-nl-C1INH, INHA, and C1INH deficiency groups. Individual results of plasma kallikrein activity after ex vivo stimulation with DXS 2.5 μg/ml is shown. Mean kallikrein activity is represented by the horizontal line for each group. Differences that were statistically significant by Wilcoxon test are shown. Panel A: all subjects. Panel B: Subjects receiving a bradykinin-targeted medication within 6 days of the sample collection were excluded.

FIG. 4. Receiver operating curve for the stimulated plasma kallikrein activity assay. The ROC curve was generated using all subjects with recurrent angioedema without urticaria who had not received a bradykinin-targeted medication within 6 days of the sample collection. The line of no-discrimination is shown as a dashed line.

FIG. 5. Recombinant human plasma kallikrein yields a linear curve that can be used to standardize the assay. Various doses of rKLKB1 were added in place of subject plasma and the activity measured as described.

FIG. 6, panels A-F: Subjects with bradykinin-mediated angioedema show cleavage of high molecular weight kininogen at a lower dose of DXS than histaminergic angioedema or non-swelling control subjects. Plasma was incubated with various doses of DXS then immunoblotted for high molecular weight kininogen under reducing conditions. The intact one-chain kininogen migrates at an apparent molecular weight of 110 kD while the cleaved light chain appears as 62 and 50 kD bands. The RFU values from the same plasma samples stimulated with DXS 2.5 μg/ml are shown. The panels are as follows: Panel A: non-swelling control; Panel B: histaminergic angioedema; Panel C: HAE-C1INH; Panel D: HAE-FXII; Panel E: HAE-U; and Panel F: INHA.

### DETAILED DESCRIPTION

Angioedema is a sudden, transient swelling of well-demarcated areas of the dermis, subcutaneous tissue, mucosa, and submucosal tissues that can occur with or without urticaria. Up to 25% of people in the US will experience an episode of urticaria or angioedema during their lifetime, and many will present to the emergency department with an acute attack. Most cases of angioedema are attributable to the vasoactive mediators histamine and bradykinin. Histamine-mediated (allergic) angioedema occurs through a type I hypersensitivity reaction, or are of unknown, non-allergic origin and are identified as Idiopathic Histaminergic Angioedema (IHA) with or without urticaria. In contrast, bradykinin-mediated (non-allergic) angioedema is iatrogenic or hereditary in origin or may be idiopathic (unknown origin) and consistently the angioedema is without urticaria.

Although their clinical presentations are similar the treatment regimen for histamine-mediated angioedema differs significantly from that for bradykinin-mediated angioedema. Thus, for example, corticosteroids, and epinephrine are effective in the management of histamine-mediated angioedema, but are ineffective in the management of bradykinin-mediated angioedema. Conversely, bradykinin-mediated angioedema can be treated with kallikrein inhibitors (e.g., ecallantide) bradykinin β2 receptor antagonists (e.g., icatibant), and the like.

In view of the very different clinical presentations and treatment regimen for histamine-mediated angioedema and bradykinin-mediated angioedema, the ability to distinguish between these types of angioedema is critical in optimizing outcomes in the acute care setting with appropriate treatment.

We have developed an assay that allows clinicians to determine whether patients with recurrent angioedema have bradykinin-mediated angioedema, e.g., thereby distinguishing bradykinin-mediated angioedema from histamine-mediated angioedema.

Bradykinin-mediated angioedema is much more likely to lead to morbidity and mortality than other forms of angioedema, and the treatment of bradykinin-mediated angioedema is distinct from other forms of angioedema.

Accordingly, in various embodiments assays are provided to distinguish bradykinin-mediated from histamine-mediated angioedema.

Additionally methods of treatment are provided where treatment regimen and/or the population to be given a particular treatment regimen is informed in part by the assays described herein.

### Identification of Bradykinin-Mediated Versus Histamine-Mediated Angioedema.

We discovered that patients with recurrent bradykinin-mediated angioedema demonstrate enhanced activation of the plasma contact system following ex vivo treatment the patients' plasma with a contact system activator (e.g., a kallikrein stimulating agent, e.g., dextran sulfate). Ex vivo stimulation of plasma with a contact system activator provides a simple, fast and reproducible readout of contact system activation that can serve as the foundation of a clinically relevant laboratory test to diagnose bradykinin-mediated angioedema.

The diagnosis of bradykinin-mediated angioedema in the setting of normal C1INH function is often difficult due to the current lack of a reliable biomarker. The stimulated kallikrein activity assays described herein accurately identified subjects with presumed bradykinin-mediated angioedema and are believed to be clinically useful in the characterization and management of angioedema patients.

The present methods are based in part, on the discovery that plasma kallikrein activity stimulated by threshold amount of a contact system activator provides a reliable biomarker that aids the diagnosis of HAE-nl-C1INH and INHA. This is in marked contrast to spontaneous plasma kallikrein amylolytic activity which, while previously reported to identify patients with HAE□nl□C1INH (Marcos et al. (2012) Ann. Allergy Asthma Immunol. 109(195-200): e2), was observed to show only a modest increase in C1NH-deficient subjects and showed no increase in HAE-nl-C1NH or INHA subjects (see, Example 1, Table 1).

Accordingly, in certain embodiments, a method of distinguishing a patient suffering bradykinin-mediated angioedema from a patient suffering from histamine-mediated angioedema is provided. In certain embodiments the method involves:
i) stimulating a plasma sample from the patient with a threshold amount of a kallikrein stimulating agent or an activator of the plasma contact system;
ii) measuring kallikrein protease activity in said sample; and
iii) identifying an increased level of kallikrein protease activity in comparison to kallikrein protease activity from a plasma sample of a normal control as indicative that the patient is suffering bradykinin-mediated angioedema; or
identifying an essentially equivalent level of kallikrein protease activity in comparison to kallikrein protease activity from a plasma sample of a normal control as indicative that the patient is suffering histamine-mediated angioedema

In various embodiments the patient will be a patient presenting with one or more symptoms of angioedema (e.g., swelling of one or more of the hands, feet, area around the eyes, lips, tongue, genitals, and/or inside of the throat, and/or difficulty breathing; and/or dizziness or fainting).

In certain embodiments, to be indicative of a bradykinin-mediated angioedema, the increased level of kallikrein protease activity in comparison to kallikrein protease activity from a plasma sample of a normal control comprises a statistically significant increased level (e.g., at a 5% (0.05) confidence level, or at a 1% (0.01) confidence level, or at a 0.1% (0.001) confidence level). In certain embodiments to be indicative of a bradykinin-mediated angioedema, the increased level of kallikrein protease activity in plasma from patients in comparison to kallikrein protease activity from a plasma sample of a normal control comprises a kallikrein activity level at least 50%, or at least 60%, or at least 70%, or at least 80%, or at least 90%, or at least 100%, or at least 1.5 fold, or at least 2 fold, or at least 3 fold, or at least 4-fold, or at least 5-fold higher than the normal healthy control level in a plasma sample stimulated with a threshold amount of a kallikrein stimulating agent or an activator of the plasma contact system. In certain preferred embodiments the increased level of kallikrein protease activity in plasma from patients in comparison to kallikrein protease activity from a plasma sample of a normal control comprises a kallikrein activity level at least 5-fold higher than the normal healthy control level in a plasma sample stimulated with a threshold amount of a kallikrein stimulating agent or an activator of the plasma contact system.

In certain embodiments, to be indicative of a histamine-mediated angioedema, the substantially equivalent level of kallikrein protease activity in comparison to kallikrein protease activity from a plasma sample of a normal healthy control comprises a statistically insignificant difference from the normal (healthy) control (e.g., at a 5% (0.05) confidence level, or at a 1% (0.01) confidence level, or at a 0.1% (0.001) confidence level). In certain embodiments, to be indicative of a histamine-mediated angioedema, the substantially equivalent level of kallikrein protease activity in comparison to the level of kallikrein protease activity from a plasma sample of a normal control comprises a kallikrein protease activity level within ±20%, or within ±15%, or within ±10%, or within ±5% of the normal healthy control level, or within a range of 5% to 20% of the level of kallikrein protease activity from a plasma sample from a normal control. In certain embodiments, to be indicative of a histamine-mediated angioedema, the substantially equivalent level of kallikrein protease activity in comparison to the level of kallikrein protease activity from a plasma sample of a normal control comprises a kallikrein protease activity level within ±20% of the level of kallikrein protease activity from a plasma sample from a normal control.

In certain embodiments the normal (healthy) control comprises a mean or median level of kallikrein protease activity determined from a population known not to have angioedema. In certain embodiments the normal control can comprise a “non-swelling” control that comprises a subject that may have an allergic reaction (e.g., asthma), but there is no swelling, hence no angioedema. In certain embodiments the population is selected to match the gender and/or age of the subject being tested.

In certain embodiments the normal control comprises a level of kallikrein protease activity from a subject determined not to have angioedema. In certain embodiments the level of kallikrein protease activity from a subject determined not to have angioedema comprises a level of kallikrein protease activity determined from the patient at a time when said subject patient is asymptomatic for angioedema (e.g., as perhaps determined in a routine blood workup). In certain embodiments the level of kallikrein protease activity from a subject determined not to have angioedema comprises a level of kallikrein protease activity determined from a subject that is not the patient.

While assay methods described herein are illustrated utilizing dextran sulfate (DXS) as a contact system activator, any known contact system activator can be used in the present assays. Illustrative, but non-limiting contact system activators include, but are not limited to, artificial negatively charged substances that activate FXII such as polyphosphates and the like.

As described in Example 1, consecutive patients with angioedema who were evaluated and followed at a single center were enrolled. Subjects were clinically phenotyped based on clinical history and response to treatment by clinicians blinded to laboratory assay results.

Plasma kallikrein activity was measured by the cleavage of the fluorometric substrate Z-Phe-Arg-AMC-HCL in plasma samples stimulated ex vivo with sub-maximal doses of dextran sulfate. This fluorometric substrate assay is illustrative and non-limiting. Other assays of plasma kallikrein activity (e.g., H-D-Pro-Phe-Arg-pNA.2HCl (Chromogenic substrate S-2302 from diapharma, and the like) are well known to those of skill in the art.

Plasma kallikrein activity was significantly increased in both HAE-nl-C1INH and INHA subjects compared to non-swelling controls and histaminergic angioedema subjects. Using a threshold cutoff based on the normal controls, bradykinin-mediated angioedema patients could be differentiated with high sensitivity and specificity.

Plasma kallikrein activity in plasma stimulated ex vivo with sub-maximal doses of dextran sulfate accurately distinguished bradykinin-from histamine-mediated angioedema as determined by clinical phenotyping. This plasma kallikrein activity assay provides a biomarker for identifying patients who may benefit from bradykinin-targeted therapy. The stimulated kallikrein activity assay allows differentiation of bradykinin-from histamine-mediated angioedema. The assay is feasible to be considered as a clinical tool for the diagnosis of bradykinin-mediated angioedema.

### Methods of Treatment.

In various embodiments the assays described herein permit identifying a patient (e.g., a patient presenting with angioedema) as having bradykinin-mediated angioedema or histamine-mediated angioedema. Depending on the type of angioedema the resulting treatment regimen will differ significantly.

Accordingly in various embodiments methods of treatment are provided where the methods involve performing the assays described herein or receiving the results of such assays to identify the subject (patient) as one having a bradykinin-mediated angioedema or a histamine-mediated angioedema. Where the patient is identified as having a bradykinin-mediated angioedema, the patient is treated for bradykinin-mediated angioedema, and where the patient is identified as having a histamine-mediated angioedema, the patient is treated for histamine-mediated angioedema. Methods of treating patients for bradykinin-mediated or histamine-mediated angioedema are known to those of skill in the art.

Treatment of Bradykinin-Mediated Angioedema.

In various embodiments treatment of bradykinin-mediated angioedema involves administering to the subject a bradykinin-targeted medication (a bradykinin-targeted therapy). In certain embodiments the bradykinin-targeted medication targets bradykinin or bradykinin receptors, or targets one or more components of a pathway involved in a bradykinin-targeted angioedema.

Historically the mainstay of emergency medical treatment for bradykinin-mediated angioedema has been IV fresh frozen plasma (FFP) and epsilon-aminocaproic acid. It is believed that FFP replaces plasma C1-INH, thereby aborting an ongoing attack. There may be, however, an increased risk of worsened swelling following administration of FFP during an acute attack, which is can be due to the concurrent replacement of both plasma proteases and substrates that are involved in the mediation of an attack.

One alternative to FFP is C1-INH replacement therapy. C1-INH replacement therapy functions to restore the missing C1-INH in patients with HAE. Berinert is a human, plasma-derived, pasteurized form of C1-INH that has been approved by the US Food and Drug Administration (FDA) for the treatment of acute abdominal, facial, and, laryngeal attacks of HAE in adult and adolescent patients.

Plasma kallikrein inhibitors can also be used in the treatment of bradykinin-mediated angioedema. In 2009, the FDA granted approval to ecallantide (Kalbitor), for the treatment of acute attacks of HAE in patients 16 years of age and older. Ecallantide is a plasma kallikrein inhibitor that is effective against attacks of HAE at any anatomic location, including abdominal/gastrointestinal, laryngeal, and peripheral attacks. Ecallantide binds to plasma kallikrein and blocks its binding site, inhibiting the conversion of HMWK to bradykinin. By directly inhibiting plasma kallikrein, ecallantide reduces the conversion of HMWK to bradykinin and thereby treats symptoms that occur during acute episodic attacks of HAE.

Bradykinin receptor antagonists can also be used in the treatment of bradykinin-mediated angioedema. The bradykinin receptor blocker icatibant is a synthetic, 10 amino acid, short-acting, and highly selective competitive bradykinin β2 receptor antagonist.

C1 esterase inhibitors (e.g., ruconest) also find utility in the treatment of bradykinin-mediated angioedema.

The foregoing bradykinin-targeted therapies are illustrative and non-limiting. Numerous other bradykinin-targeted therapies will be known to those of skill in the art and used in the methods described herein.

Treatment of Histamine-Mediated Angioedema.

In various embodiments treatment of histamine-mediated angioedema involves administering to the subject a histamine-targeted medication (a histamine-targeted therapy). In certain embodiments the histamine-targeted medication targets histamine release or histamine receptors, or targets one or more components of a pathway involved in a histamine-targeted angioedema. In certain embodiments the histamine-targeted medication comprises one or more agents selected from the group consisting of H1 receptor antagonists and drugs that are not histamine targeted but that are needed due to the adrenergic pathways and inflammatory response that become activated by histamine release (e.g., epinephrine, an antihistamine, a β2 agonist, a corticosteroid. Also contemplated are mast-cell targeted medications to prevent release of histamine.

In certain embodiments the treatment of histamine-mediated angioedema involves the treatment of anaphylaxis which can involve administration of epinephrine and/or intubation as required.

In certain embodiments the histamine targeted medication (histamine-targeted therapy) comprises one or more agents selected from the group consisting of epinephrine, an antihistamine, a β2 agonist (e.g., albuterol), a corticosteroid (e.g., methylprednisolone), and a mast-cell targeted medication (e.g., a leukotriene modifier or omalizumab).

The foregoing histamine-targeted therapies are illustrative and non-limiting. Numerous other histamine-targeted therapies will be known to those of skill in the art and

### EXAMPLES

The following examples are offered to illustrate, but not to limit the claimed invention.

### Example 1

In this Example, we describe a simple assay that discriminates histamine- from bradykinin-mediated angioedema. This assay is based on activation of plasma kallikrein following ex vivo stimulation of the contact system in plasma. Utilizing a threshold dose of the stimulator, we could differentiate subjects with bradykinin-mediated from IHA without urticaria. This assay identifies patients with possible HAE-unknown or INHA who could benefit from a trial of a bradykinin-targeted medication. By segregating these patients from IHA, this assay also facilitates efforts to identify the underlying mechanisms of bradykinin-mediated angioedema with normal C1INH.

### Materials and Methods.

Human Subjects.

This study was approved by the Institutional Review Board and Human Research Protections Program of the University of California (UC) San Diego. Consecutive patients presenting with angioedema without urticaria to the US Hereditary Angioedema Association (HAEA) Angioedema Center at UC San Diego were informed of the study and given the opportunity to participate. Subjects were required to be age 14 or above. All study subjects (and parents for minors) signed informed consent/assent forms prior to any study procedures. Control subjects included normal healthy volunteers as well as patients without angioedema or urticaria.

Clinical Phenotyping:

All subjects were clinically phenotyped based on clinical history, standard laboratory values, and response to treatment by blinded investigators (SC, MR, BZ). Using the consensus guidelines (16,25), subjects were assigned to seven different groups: 1) HAE-C1INH; 2) ACID; 3) HAE-XII; 4) HAE-unknown; 5) INHA; 6) IHA; and 7) non-swelling control (NSC). Subjects classified as potentially having INHA met the same criteria as HAE-unknown but did not have a positive family history of angioedema.

Subjects with a diagnosis of HAE-unknown or INHA were further classified based on probability using two pre-defined criteria not in the consensus guidelines: 1) history of rapid and durable responses to a bradykinin-targeted medication; and 2) either predominantly documented visible angioedema; or in patients with predominant abdominal symptoms, evidence of bowel wall edema documented by CT or MRI. To be classified as high probability, subjects needed to meet both additional criteria. A negative response to either of these reduced the probability to moderate, and negative responses to both reduced the probability to low.

Blood Drawing and Processing:

At the time of blood sampling, history of recent angioedema attacks and medication use was recorded. Blood was collected into plastic vacutainer tubes containing EDTA or citrate (Becton Dickinson, Franklin Lakes, N.J.) after removing the tourniquet and discarding at least the first 5 ml of blood. Tubes were kept at room temperature until processed within 15 minutes of the draw then centrifuged at 2,000×g for 10 min at room temperature. Plasma was aliquoted into Corning 1.2 mL internal threaded polypropylene cryogenic vials, immediately snap frozen in liquid nitrogen, and stored at −80° C. until analyzed. Plasma aliquots were thawed a maximum of two times. All assays were done by ML-M or JH.

Reagents and Chemicals:

The following reagents and chemicals were purchased: dextran sulfate (DXS) and dimethyl sulfoxide anhydrous (DMSO) from Sigma-Aldrich, St. Louis, Mo.; Z-Phe-Arg-AMC-HCl from BACHEM, Torrance, Calif.; recombinant human plasma kallikrein (rKLKB1, Lot NVH0215121) from R&D Systems, Minneapolis, Minn.; human pre-kallikrein deficient citrated plasma from Aniara, West Chester, Ohio; pre-cast TGX Mini Protean SDS-PAGE Gels and 2× Laemmli premixed buffer from Bio-Rad, Hercules, Calif.

Fresh DXS stock was prepared every 4 months at 20 mg/ml in distilled water, sterilized by filtration, and stored at 4° C. until used. The DXS was diluted in 4× assay buffer (see below) within 2 hours of the assay. Z-Phe-Arg-AMC-HCl was dissolved in DMSO to 90 mM stock solution and stored at −20° C. Ten minutes prior to the assay, the stock Z-Phe-Arg-AMC-HCl was diluted to the final concentration with filtered deionized H₂O, then kept at room temperature in the dark until used.

Plasma Kallikrein Fluorometric Activity Assay:

Plasma aliquots were thawed in a 37° C. water bath for 4 minutes immediately prior to performing the assay. The assay was performed in 96-well plates (NUNC Black Maxisorp) with a total volume of 100 μl per well with 2-3 replicates per sample. Each aliquot included a negative control (no DXS) to calculate spontaneous kallikrein activity. The plates were prepared by adding the reagents within 6 minutes in the following order: 25 μl of 4× assay buffer (320 mM NaCl, 200 mM Tris-HCl, pH 7.8) with or without DXS (0, 1.25, 2.5, or 5 μg/ml final concentration); 25 μl of EDTA plasma or rKLK1 (varying concentrations); and 50 μl of substrate (100 μM of Z-Phe-Arg-AMC-HCl final concentration). The plates were immediately loaded into the spectrophotometer (Synergy H1 Reader, BioTek, Winooski, Vt., USA stabilized for 30 min at 37° C. prior to the assay). Assay parameters were shaking the plate for 5 seconds followed by measurement of fluorescence (excitation 360 nm, emission 460 nm) every 60 seconds for 30 minutes. Results were calculated as the slope of relative fluorescence units (RFU) over the 30-minute assay, subtracting spontaneous kallikrein activity at each point.

Plasma High Molecular Weight Kininogen (HMWK) Immunoblotting:

Citrated plasma samples (10 μl) were incubated with varying concentrations of DXS (10 μl) for 30 min. at 37° C., and the stimulation stopped by dilution with an equal volume of 2× Laemmli loading buffer. Immunoblotting for HMWK was performed under both reducing and non-reducing conditions as previously described (26).

Data and Statistical Analysis:

GraphPad PRISM 7.0a software for MAC was used to perform linear regression analysis and slope calculations (La Jolla, Calif., USA). JMP Statistical Analysis Software (SAS) was used for data analysis (Cary, N.C., USA). Group comparisons used the Wilcoxon signed rank test. Differences with a p value<0.05 were considered significant.

### Results.

Subject Demographics and Phenotyping.

A total of 154 research subjects were enrolled. The final clinical phenotype for each subject reflected the initial consultative evaluation as well as information collected on clinical follow-up. Every subject was independently phenotyped by at least two expert angioedema clinicians who were blinded to the experimental results. In the few cases where there was a difference in initial phenotype decision, subjects' clinical data was reviewed by all 3 experts until a unanimous consensus was reached. Random codes were broken and experimental assay results linked to the subjects only after all the clinical phenotyping was finalized.

Of 154 subjects, 37 were excluded because they were found to have concomitant urticaria. Subjects considered to have low probability of HAE-nl-C1INH or INHA (n=7) were also excluded. Eight subjects were felt to have features of both bradykinin-mediated and histamine-mediated angioedema. These subjects were classified in a separate group, identified as intermediate, and excluded from the analysis. Additional excluded subjects were as follows: 3 subjects diagnosed with ACE inhibitor associated angioedema; 2 subjects with complex seizure disorders requiring use of multiple drugs; 2 subjects whose plasma was not processed in a timely fashion; and 5 subjects adjudicated not to have angioedema. This left 90 subjects in the analyzed cohort. Subject demographics of the analyzed cohort are shown in Table 1.

TABLE 1
Spontaneous and DXS-stimulated kallikrein activity

| Group | N | F | Age (yr ± SD) | Spontaneous |
|---|---|---|---|---|
| Non-swelling | 14 | 64.3% | 43.4 ± 15.5 | 1.8 ± 1.8 |
| Histaminergic AE | 24 | 37.5% | 52.5 ± 15.9 | 7.5 ± 4.1 |
| C1INH deficiency | 9 | 66.7% | 45.4 ± 16.7 | 160.7 ± 77.9^{‡} |
| HAE-nl-C1INH | 20 | 100.0% | 46.8 ± 13.4 | 9.3 ± 4.4 |
| INHA | 23 | 91.3% | 38.6 ± 14.5 | 7.1 ± 4.7 |

| | DXS Stimulation | | |
|---|---|---|---|
| Group | 1.25 μg/ml | 2.5 μg/ml | 5 μg/ml |
| Non-swelling | 34.4 ± 8.8 | 170.8 ± 45.8 | 2,910.2 ± 566.8 |
| Histaminergic AE | 21.6 ± 4.0 | 133.0 ± 29.8 | 3,132.6 ± 558.9 |
| C1INH deficiency | 348.3 ± 151.4^{‡} | 2,479.9 ± 437.2^{‡} | 8,656.8 ± 1,479.1^{‡} |
| HAE-nl-C1INH | 123.6 ± 72.4° | 1,804.3 ± 597.8^{‡} | 7,097.2 ± 742.3^{‡} |
| INHA | 212.8 ± 167.1 | 1,578.9 ± 371.2^{‡} | 6,136.6 ± 627.0^{‡} |

RFU/min (mean ± SEM)
°p < 0.05 compared to histaminergic AE
^{‡}p < 0.01 compared to histaminergic AE

Spontaneous kallikrein activity does not distinguish HAE-nl-C1INH or INHA from IHA. Spontaneous plasma kallikrein amidolytic activity has been reported to identify patients with HAE-nl-C1INH 21. As shown in Table 1, we observed a modest increase in spontaneous kallikrein activity only in the C1INH-deficient subjects. We also failed to detect any increase in spontaneous kallikrein activity in HAE-nl-C1INH or INHA subjects who were actively swelling or had experienced an angioedema attack within 1 day of the blood draw.

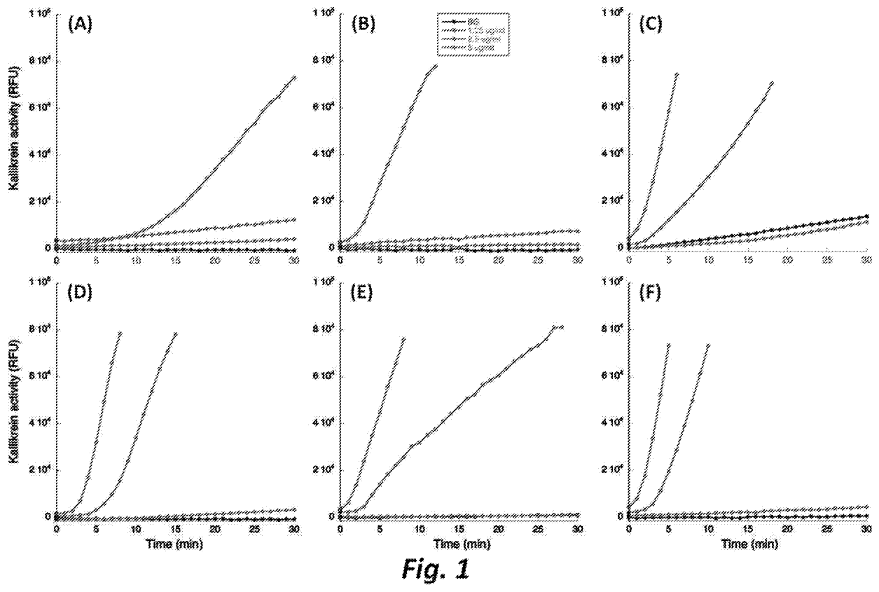

Key features of the threshold-stimulated kallikrein (TSK) assay. We investigated whether plasma kallikrein activity stimulated by a minimal threshold dose of DXS could identify bradykinin-mediated angioedema subjects. Ten μg/ml DXS stimulated maximal kallikrein activity in all subjects, and failed to discriminate between the groups. FIG. 1 shows kallikrein activity in 4 representative subjects (HAE-C1INH, HAE-nl-C1INH, IHA, and NSC) stimulated with 0, 1.25, 2.5, or 5 μg/ml DXS. Five μg/ml DXS gave a near maximal response but failed to clearly discriminate between the subjects. After 2.5 μg/ml DXS, kallikrein activity was clearly increased in the HAE-C1INH, INHA and HAE-nl-C1INH subjects compared to the IHA and NSC subjects. At 1.25 μg/ml DXS and below, only the HAE-C1INH subject showed significant kallikrein activity.

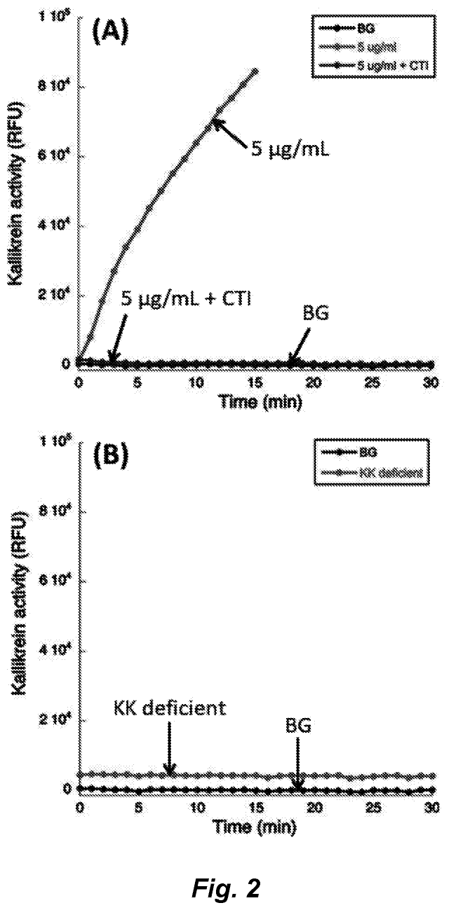

We investigated the specificity and reproducibility of the kallikrein assay. Because the fluorometric substrate used in our assay could potentially be cleaved by other proteases, we assessed the dependence of the cleavage on activation of the plasma contact system. We observed no RFU over the 30-minute assay when prekallikrein deficient plasma was stimulated with 1.25, 2.5 or 5 μg/ml DXS (FIG. 2A). Similarly, DXS stimulated cleavage of the substrate was completely abrogated in normal plasma by pre-incubation with CTI, a potent and highly specific FXII inhibitor (FIG. 2B). These results confirm that the DXS-stimulated activity measured in our assay was FXII- and plasma kallikrein-dependent.

We assessed the reproducibility of the assay both in multiple replicates performed in a single assay as well as in multiple aliquots from the same subject analyzed on different days. The average variance of multiple replicates of the same sample run on the same day averaged 7.0%. Variance of the plasma sample from 7 different subjects run on separate days was 14.5%.

The TSK assay distinguishes subjects with HAE-nl-C1INH or INHA from IHA. We analyzed kallikrein activity in the entire cohort of subjects using plasma samples that were stimulated with DXS at 0, 1.25, 2.5, and 5.0 μg/ml. Table 1 shows that 2.5 and 5.0 μg/ml DXS provided significant separation in kallikrein activity between the presumed bradykinin-mediated angioedema subjects (HAE-C1INH, ACID, HAE-nl-C1INH, and INHA) and non-bradykinin-mediated angioedema subjects (IHA and NSC).

Sensitivity and specificity of the stimulated-kallikrein activity using 2.5 or 5.0 μg/ml DXS for the diagnosis of HAE-nl-C1INH or INHA was assessed based on our clinical phenotyping utilizing the kallikrein activity measured in the NSC group (mean—170.8; SD—171.4). We performed a sensitivity analysis, using the mean NSC kallikrein activity plus either 1, 2, or 3 standard deviations (75-99.75 confidence interval) as the upper limit for kallikrein activity. The sensitivity analysis was performed using four subject populations: 1) entire cohort; 2) entire cohort minus HAE-U or INHA subjects who had received a bradykinin targeted medication within the prior 6 days; 3) all subjects minus the C1INH deficient subjects; and 4) all subjects minus the C1INH deficient subjects as well as subjects who had received a bradykinin targeted medication within the prior 6 days.

Tables 2 and 3 show the predictive value for the kallikrein assay after stimulating with 2.5 μg/ml (Table 2) and after stimulating with 5.0 μg/ml DXS (Table 3). The positive predictive value (PPV), negative predictive value (NPV), and odds ratio (OR) are all considerably higher after stimulation with 2.5 μg/ml DXS compared to 5.0 μg/ml DXS. Therefore, we concentrated on the 2.5 μg/ml DXS results. As expected, the sensitivity decreases and the specificity increases as the stringency of the upper limit of normal is increased. The PPV ranged from (80-100%) while the NPV was more variable (63.33-89.47%). Excluding subjects who had recently received a bradykinin-targeted medication improved the NPV (85.71-89.47%), suggesting that the medication may have interfered with the assay.

TABLE 2
Sensitivity analysis of predictive value of the TSK assay for the diagnosis
of HAE and INHA. Dextran sulfate 2.5 μg/ml.

| Population | Threshold SD^{‡} | True “+” | False “+” | True “−” | False “−” | Sensitivity |
|---|---|---|---|---|---|---|
| All | 1 | 35 | 4 | 34 | 17 | 67.31% |
| | 2 | 33 | 2 | 36 | 19 | 63.46% |
| | 3 | 30 | 0 | 38 | 22 | 57.69% |
| All subjects minus | 1 | 25 | 4 | 34 | 4 | 86.21% |
| recently treated | 2 | 24 | 2 | 36 | 6 | 79.31% |
| | 3 | 23 | 0 | 38 | 6 | 79.31% |
| All minus C1INH | 1 | 26 | 4 | 34 | 17 | 60.47% |
| deficient | 2 | 24 | 2 | 36 | 19 | 55.81% |
| | 3 | 21 | 0 | 38 | 22 | 48.84% |
| All minus C1INH | 1 | 16 | 4 | 34 | 4 | 80.00% |
| deficient and | 2 | 14 | 2 | 36 | 6 | 70.00% |
| recently treated | 3 | 14 | 0 | 38 | 6 | 70.00% |
| Population | Specificity | PLR* | NLR | OR | PPV | NPV |
| All | 89.47% | 6.39 | 0.37 | 17.50 | 89.74% | 66.67% |
| | 94.74% | 12.06 | 0.39 | 31.26 | 91.67% | 65.45% |
| | 100.00% | >100° | 0.42 | >100° | 100.00% | 63.33% |
| All subjects minus | 89.47% | 8.19 | 0.15 | 53.13 | 86.21% | 89.47% |
| recently treated | 94.74% | 15.07 | 0.22 | 69.00 | 92.00% | 85.71% |
| | 100.00% | >100° | 0.21 | >100° | 100.00% | 86.36% |
| All minus C1INH | 89.47% | 5.74 | 0.44 | 13.00 | 86.67% | 66.67% |
| deficient | 94.74% | 10.60 | 0.47 | 22.74 | 92.31% | 65.45% |
| | 100.00% | >100° | 0.51 | >100° | 100.00% | 63.33% |
| All minus C1INH | 89.47% | 7.60 | 0.22 | 34.00 | 80.00% | 89.47% |
| deficient and | 94.74% | 13.30 | 0.32 | 42.00 | 87.50% | 85.71% |
| recently treated | 100.00% | >100° | 0.30 | >100° | 100.00% | 86.36% |

^{‡}Upper limit of normal range calculated as NSC group mean value plus the indicated number of standard deviations
*Abbreviations used: PLR = positive likelihood ratio; NLR = negative likelihood ratio; OR = odds ratio; PPV = positive predictive value; NPV = negative predictive value
°Cannot calculate value due to infinite result

TABLE 3
Sensitivity analysis of predictive value of the TSK assay for the diagnosis
of HAE and INHA. Dextran sulfate 5.0 μg/ml

| Population | Threshold SD^{‡} | True “+” | False “+” | True “−” | False “−” | Sensitivity |
|---|---|---|---|---|---|---|
| All | 1 | 38 | 11 | 27 | 14 | 73.08% |
| | 2 | 25 | 3 | 35 | 27 | 48.08% |
| | 3 | 10 | 0 | 38 | 42 | 19.23% |
| All subjects | 1 | 23 | 11 | 27 | 6 | 79.31% |
| minus recently | 2 | 17 | 3 | 35 | 12 | 58.62% |
| treated | 3 | 8 | 0 | 38 | 21 | 27.59% |
| All minus | 1 | 31 | 11 | 27 | 12 | 72.09% |
| C1INH | 2 | 21 | 3 | 35 | 22 | 48.84% |
| deficient | 3 | 6 | 0 | 38 | 37 | 13.95% |
| All minus | 1 | 16 | 11 | 27 | 4 | 80.00% |
| C1INH | 2 | 13 | 3 | 35 | 7 | 65.00% |
| deficient and | 3 | 4 | 0 | 38 | 16 | 20.00% |
| recently treated | | | | | | |
| Population | Specificity | PLR* | NLR | OR | PPV | NPV |
| All | 71.05% | 2.52 | 0.38 | 6.66 | 77.55% | 65.85% |
| | 92.11% | 6.09 | 0.56 | 10.80 | 89.29% | 56.45% |
| | 100.00% | >100° | 0.81 | >100° | 100.00% | 47.50% |
| All subjects | 71.05% | 2.74 | 0.29 | 9.41 | 67.65% | 81.82% |
| minus recently | 92.11% | 7.43 | 0.45 | 16.53 | 85.00% | 74.47% |
| treated | 100.00% | >100° | 0.72 | >100° | 100.00% | 64.41% |
| All minus | 71.05% | 2.49 | 0.39 | 6.34 | 73.81 | 69.23 |
| C1INH | 92.11% | 6.19 | 0.56 | 11.14 | 87.50% | 61.40% |
| deficient | 100.00% | >100° | 0.86 | >100° | 100.00% | 50.67% |
| All minus | 71.05% | 2.76 | 0.28 | 9.82 | 59.26% | 87.10% |
| C1INH | 92.11% | 8.23 | 0.38 | 21.67 | 83.33% | 21.67% |
| deficient and | 100.00% | >100° | 0.80 | >100° | 100.00% | 70.37% |
| recently treated | | | | | | |

^{‡}Upper limit of normal range calculated as NSC group mean value plus the indicated number of standard deviations
*Abbreviations used: PLR = positive likelihood ratio; NLR = negative likelihood ratio; OR = odds ratio; PPV = positive predictive value; NPV = negative predictive value
°Cannot calculate value due to infinite result

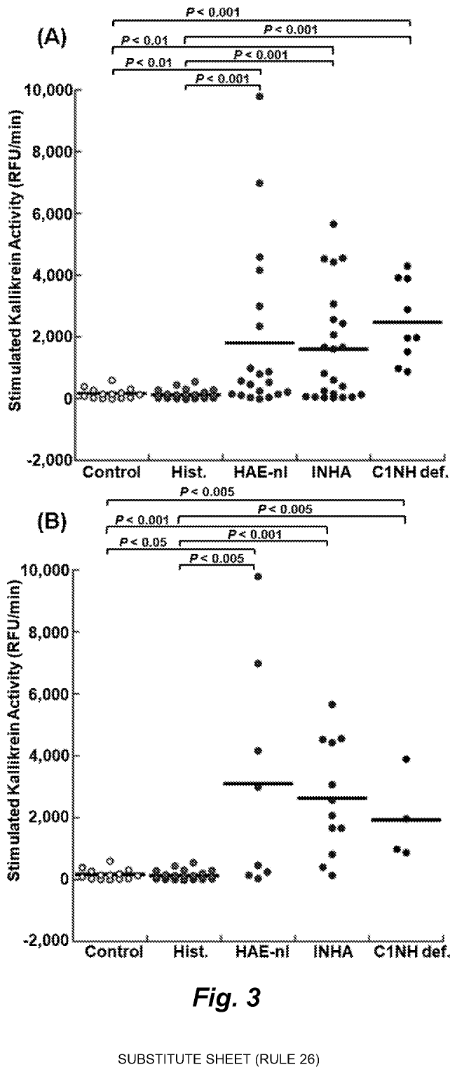

Individual 2.5 μg/ml DXS-stimulated kallikrein activity is shown in FIG. 3A while FIG. 3B shows the activity in subjects who had not recently received a bradykinin targeted medication. In every case, HAE-C1INH, HAE-nl-CINH and INHA groups had significantly elevated kallikrein activity compared to IHA and NSC groups. No significant differences were seen between the HAE-U, INHA and C1INH deficient groups. Since our HAE-nl-C1INH and INHA groups are predominantly female, we asked whether there was a gender difference in the assay. We found no difference in the 2.5 μg/ml DXS-stimulated kallikrein activity between females versus males in our IHA and NSC groups.

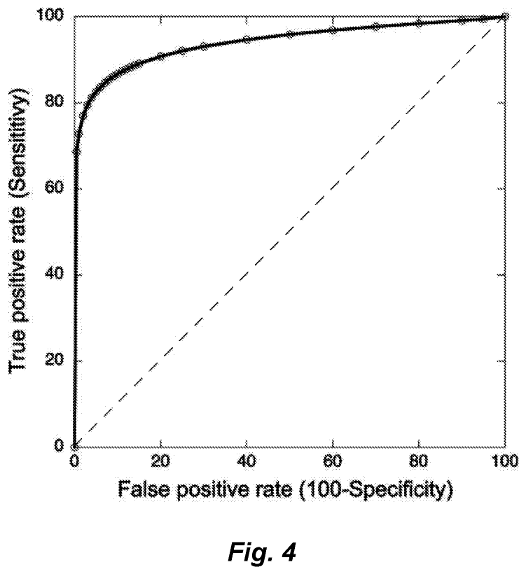

Receiver operator curve (ROC) analysis is a standard method to evaluate the value of a diagnostic test. We evaluated the TSK assay for the diagnosis of recurrent angioedema without urticaria in subjects who had not recently received bradykinin targeted treatment as this represents the clinical scenario for which the test would be most useful. As shown in FIG. 4, the kallikrein assay showed excellent characteristics as a diagnostic test based on area-under-the curve >0.91 in the ROC analysis.

DXS Stimulated Kallikrein Activity can be Modeled Using rKLKB1.

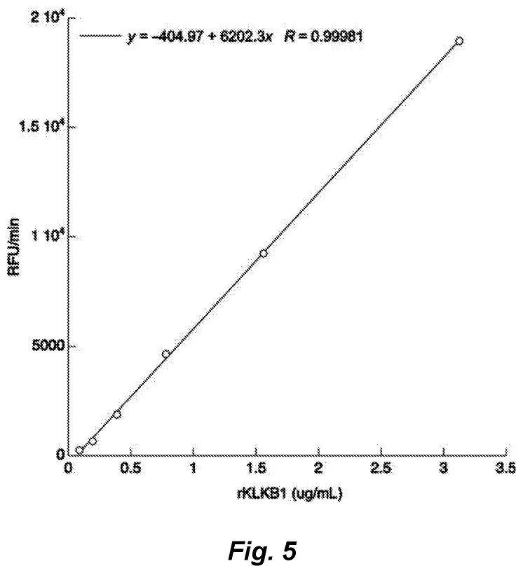

We constructed standard curves of kallikrein activity using rKLKB1, performed in an identical manner to the TSK assay except that varying amounts of rKLKB1 were added in place of plasma. FIG. 5 shows the linear characteristics of the rKLKB1 activity in this assay. By interpolating the mean kallikrein activity, we could estimate that the mean amount of kallikrein activity stimulated by 2.5 μg/ml of DXS in the studied groups were as follows: HAE-nl-C1INH, 0.37 μg/ml; INHA, 0.29 μg/ml; IHA and NSC, 0.09 μg/ml; and C1INH deficiency, 0.50 μg/ml.

Increased TSK Activity Correlates with Increased Susceptibility to Ex Vivo HA WK Cleavage.

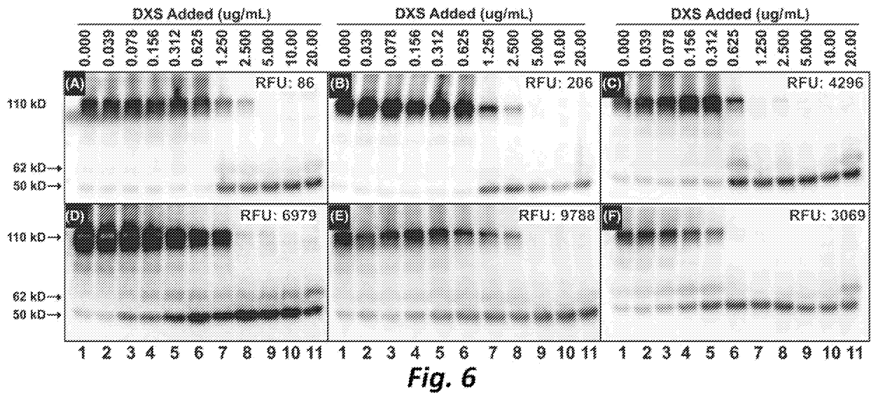

In a limited number of subjects, we assessed whether threshold DXS-stimulated kallikrein activity also tracked with enhanced susceptibility to DXS-induced HMWK cleavage. FIG. 6 shows reduced HMWK immunoblots of plasmas from 6 representative subjects (A, NSC; B, IHA; C, HAE-XII; D, HAE-CHNH; E, HAE-U; and F, INHA) that were stimulated with various doses of DXS. The kallikrein activity at 2.5 μg/ml DXS is also shown for each sample. Plasmas that displayed an increased kallikrein activity demonstrated corresponding cleavage of HMWK at lower doses of DXS, supporting the physiologic relevance of the kallikrein activity. Non-reduced HMWK immunoblots gave similar results.

### Discussion.

Current consensus guidelines for the diagnosis of HAE-U and INHA ultimately depend on excluding alternative diagnoses. With the exception of the relatively small percentage of HAE-nl-C1INH patients with identified mutations, there are no diagnostic tests that can confirm the diagnosis of HAE-nl-C1INH or INHA. The absence of a confirmative assay causes substantial clinical problems, involving both under- and over-diagnosis of these conditions. Under-diagnosis carries an attendant risk of morbidity and mortality due to the failure to treat bradykinin-mediated angioedema attacks with effective drugs. Over-diagnosis leads to inappropriate use of costly medications and the failure to pursue other treatments that could ameliorate symptoms. Both under- and over-diagnosis cause considerable confusion for patients and physicians. In this report, we describe a TSK assay that provides a new tool to positively identify patients with HAE-U and INHA.

Clinical phenotyping was critical to the analysis and utilized stringent criteria. First, we utilized current consensus guidelines. Second, phenotype was determined by comprehensive clinical evaluation and chart review by at least two physicians with expertise in angioedema. Third, subjects were subdivided into high, moderate or low probability categories based on additional strict criteria. Fourth, in the rare cases of disagreement about diagnosis or probability, all three expert clinicians reviewed the data together until a consensus was reached. Clinicians were blinded to the assay results until phenotyping was completed.

Two groups were excluded from further analysis. The first consisted of 7 subjects classified as HAE-U low probability. These subjects reported predominantly episodic abdominal pain without documented imaging showing bowel wall edema and did not report clear benefit from treatment with a bradykinin pathway acting drug. While these subjects appeared to meet the consensus criteria for HAE-U, our clinical impression was that they did not have true bradykinin-mediated angioedema. When the kallikrein data was unblinded, we found that each of these subjects had normal kallikrein activity—corroborating the clinical impression. The second group consisted of 8 subjects who appeared to have a mixed phenotype with elements of both histamine-mediated and bradykinin-mediated angioedema. In general, they appeared to have strong evidence of bradykinin-mediated angioedema but also reported modest benefit from high dose antihistamine treatment. Interestingly, kallikrein activity in this group was elevated in 5 of the 8 subjects, suggesting a possible mixed pathophysiology in at least some of these individuals.

Increased TSK activity was highly correlated with clinical phenotype. Most new patients with recurrent angioedema without urticaria will not have been exposed to a bradykinin pathway acting drug. In these patients, we found a PPV ranging from 85.71 to 89.47% and a NPV ranging from 85.71 to 89.47% depending on the threshold cutoff. The high specificity of this assay suggests that patients with an elevated kallikrein activity should be considered eligible for a therapeutic trial with a bradykinin targeted medication. The converse situation where a patient suspected of having HAE-U or INHA has a normal kallikrein activity should alert the physician to the possibility that this patient might not have a bradykinin-mediated angioedema. Trials of alternative mast cell targeted medications such as leukotriene modifiers or omalizumab should be considered prior to any trial of a bradykinin targeted medication.

The TSK assay should be adaptable to clinical practice. Samples can be stored at −80° C. for at least a month prior to assay, the assay itself is relatively simple, fast, and rKLKB1 can be used to standardize the assay allowing comparisons between results obtained at different times or locations. The assay is entirely dependent on activation of the plasma contact system and is highly reproducible. The receiver operating characteristic curve of the assay appears excellent, supporting the potential clinical utility of the assay.

Limitations of the assay include the need to collect the blood sample using good technique with rapid processing of the sample. It is important to note that recently administered bradykinin-targeted medications appear to interfere with the assay.

Several other assays have been reported to provide useful information in diagnosing HAE-U or INHA. Spontaneous kallikrein chromogenic activity was reported to identify HAE-nl-C1INH patients (21). We failed to confirm any increase in spontaneous kallikrein activity in our HAE-nl-C1INH subjects. Decreased levels of PAI-2 in patients with HAE-XII and HAE-U were reported (27), however subsequent reports failed to confirm the initial observation (28,29).

In conclusion, we report a novel biomarker for the diagnosis of HAE-U and INHA. The TSK assay shows high specificity and positive likelihood ratio for presumed bradykinin-mediated angioedema. The lack of an existing diagnostic test or biomarker for HAE-U or INHA has caused substantial difficulties for patients and physicians resulting in both the failure to treat patients with severe life-threatening angioedema with appropriate medications as well as inappropriate use of costly medications. The inability to establish an accurate diagnosis also severely hampers the ability to conduct genetic studies or therapeutic trials due to the non-homogeneity of the population. The TSK assay can be adapted for clinical use and can improve patient care while aiding future studies aimed at identifying specific causes of bradykinin-mediated angioedema.

### Abbreviations

ACID—acquired C1 inhibitor deficiency

C1INH— C1 inhibitor

CTI—corn trypsin inhibitor

DMSO—dimethyl sulfoxide anhydrous,

DXS—dextran sulfate

FXII—factor XII

HAE—hereditary angioedema

HAEA—hereditary angioedema association

HAE-C1INH—hereditary angioedema due to C1 inhibitor deficiency

HAE-FXII—hereditary angioedema due to factor F12 mutation

HAE-nl-C1INH hereditary angioedema with normal C1 inhibitor

HAE-U—hereditary angioedema unknown type

HMWK—high molecular weight kininogen

INH—idiopathic histaminergic angioedema

INHA—idiopathic non-histaminergic angioedema

NSC—non-swelling controls

NPV—negative predictive value

OR—odds ratio

PPV—positive predictive value

RFU—relative fluorescence units

ROC—Receiver operator curve

rKLKB1—recombinant human plasma kallikrein

TSK—threshold stimulated kallikrein

### REFERENCES

1 Zuraw B L. Clinical practice. Hereditary angioedema. N Engl J Med 2008; 359:1027-36.
2 Zuraw B L, Bernstein J A, Lang D M, et al. A focused parameter update: Hereditary angioedema, acquired C1 inhibitor deficiency, and angiotensin-converting enzyme inhibitor-associated angioedema. The Journal of allergy and clinical immunology 2013; 131:1491-3 e25.
3 Davis A E, 3rd, Lu F, Mejia P. C1 inhibitor, a multi-functional serine protease inhibitor. Thromb Haemost 2010; 104:886-93.
4 Walford H H, Zuraw B L. Current update on cellular and molecular mechanisms of hereditary angioedema. Ann Allergy Asthma Immunol 2014: 112:413-8.
5 Christiansen S C, Zuraw B L. Laboratory Approaches for Assessing Contact
System Activation. Immunol Allergy Clin North Am 2017; 37:527-39.
6 Kaplan A P, Joseph K. Pathogenic mechanisms of bradykinin mediated diseases: dysregulation of an innate inflammatory pathway. Adv Immunol 2014; 121:41-89.
7 Bork K, Barnstedt S E, Koch P, Traupe H. Hereditary angioedema with normal C1-inhibitor activity in women. Lancet 2000; 356:213-7.
8 Binkley K E, Davis A, 3rd. Clinical, biochemical, and genetic characterization of a novel estrogen-dependent inherited form of angioedema. J Allergy Clin Immunol 2000; 106:546-50.
9 Dewald G, Bork K. Missense mutations in the coagulation factor XII (Hageman factor) gene in hereditary angioedema with normal C1 inhibitor. Biochem Biophys Res Commun 2006; 343:1286-9.
10 Bork K, Wulff K, Meinke P, Wagner N, Hardt J, Witzke G. A novel mutation in the coagulation factor 12 gene in subjects with hereditary angioedema and normal C1-inhibitor. Clin Immunol 2011; 141:31-5.
11 Kiss N, Barabas E, Varnai K, et al. Novel duplication in the F12 gene in a patient with recurrent angioedema. Clin Immunol 2013; 149:142-5.
12 Bork K, Wulff K, Hardt J, Witzke G, Lohse P. Characterization of a partial exon 9/intron 9 deletion in the coagulation factor XII gene (F12) detected in two Turkish families with hereditary angioedema and normal C1 inhibitor. Haemophilia 2014; 20:e372-5.
13 Bork K, Wulff K, Hardt J, Witzke G, Staubach P. Hereditary angioedema caused by missense mutations in the factor XII gene: Clinical features, trigger factors, and therapy. J Allergy Clin Immunol 2009; 124:129-34.
14 Bafunno V, Firinu D, D'Apolito M, et al. Mutation of the angiopoietin-1 gene (ANGPT1) associates with a new type of hereditary angioedema. J Allergy Clin Immunol 2017; in press.
15 Bork K, Wulff K, Steinmuller-Magin L, et al. Hereditary angioedema with a mutation in the plasminogen gene. Allergy 2017; in press.
16 Zuraw B L, Bork K, Binkley K E, et al. Hereditary angioedema with normal C1 inhibitor function: consensus of an international expert panel. Allergy and asthma proceedings 2012; 33 Suppl 1:S145-56.
17 Bork K, Wulff K, Witzke G, Stanger C, Lohse P, Hardt J. Antihistamine-resistant angioedema in women with negative family history: estrogens and F12 gene mutations. Am J Med 2013; 126:1142 e9-14.
18 Bjorkqvist J, de Maat S, Lewandrowski U, et al. Defective glycosylation of coagulation factor XII underlies hereditary angioedema type III. J Clin Invest 2015; 125:3132-46.
19 de Maat S, Bjorkqvist J, Suffritti C, et al. Plasmin is a natural trigger for bradykinin production in patients with hereditary angioedema with factor XII mutations. J Allergy Clin Immunol 2016.
20 Ghannam A, Defendi F, Charignon D, et al. Contact system activation in patients with HAE and normal C1 inhibitor function. Immunol Allergy Clin North Am 2013; 33:513-33.
21 Defendi F, Charignon D, Ghannam A, et al. Enzymatic assays for the diagnosis of bradykinin-dependent angioedema. PLoS One 2013; 8:e70140.
22 Boccon-Gibod I, Bouillet L. Safety and efficacy of icatibant self-administration for acute hereditary angioedema. Clin Exp Immunol 2012; 168:303-7.
23 Marcos C, Lopez Lera A, Varela S, Linares T, Alvarez-Eire M G, Lopez-Trascasa M. Clinical, biochemical, and genetic characterization of type III hereditary angioedema in 13 Northwest Spanish families. Ann Allergy Asthma Immunol 2012; 109:195-200 e2.
24 Cronin J A, Maples K M. Treatment of an acute attack of type III hereditary angioedema with ecallantide. Ann Allergy Asthma Immunol 2012; 108:61-2.
25 Cicardi M, Aberer W, Banerji A, et al. Classification, diagnosis, and approach to treatment for angioedema: consensus report from the Hereditary Angioedema International Working Group. Allergy 2014; 69:602-16.
26 Berrettini M, Lammle B, White T, et al. Detection of in vitro and in vivo cleavage of high molecular weight kininogen in human plasma by immunoblotting with monoclonal antibodies. Blood 1986; 68:455-62.
27 Joseph K, Tholanikunnel B G, Wolf B, Bork K, Kaplan A P. Deficiency of plasminogen activator inhibitor 2 in plasma of patients with hereditary angioedema with normal C1 inhibitor levels. J Allergy Clin Immunol 2016; 137:1822.
28 Marlu R, Deroux A, Du-Thanh A, Boccon-Gibod I, Launay D, Bouillet L. Normal PAI-2 level in French FXII-HAE patients. J Allergy Clin Immunol 2017; 139:1719-20.
29 Joseph K, Constantino-Silva R N, Grumach A S, et al. Reply. J Allergy Clin Immunol 2017; 139:1720-1.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of distinguishing a patient suffering bradykinin-mediated angioedema from a patient suffering from histamine-mediated angioedema, comprising:
stimulating a plasma sample from the patient with a threshold amount of a kallikrein stimulating agent or an activator of the plasma contact system;
measuring kallikrein protease activity in said sample; and
identifying an increased level of kallikrein protease activity in comparison to kallikrein protease activity from a plasma sample of a normal control as indicative that the patient is suffering bradykinin-mediated angioedema; or
identifying an essentially equivalent level of kallikrein protease activity in comparison to kallikrein protease activity from a plasma sample of a normal control as indicative that the patient is suffering histamine-mediated angioedema.

2. The method of claim 1, wherein said patient is a patient presenting with one or more symptoms of angioedema.

3. The method of claim 2, wherein said symptoms of angioedema comprise one or more symptoms selected from:
swelling of one or more of the hands, feet, area around the eyes, lips, tongue, genitals, and/or inside of the throat; and/or
difficulty breathing; and/or dizziness or fainting.

4. The method according to any one of claims 1-3, wherein said increased level of kallikrein protease activity in comparison to kallikrein protease activity from a plasma sample of a normal control comprises a statistically significant increased level at a 5%, or at a 1% confidence level.

5. The method according to any one of claims 1-4, wherein said increased level of kallikrein protease activity comprises a kallikrein activity level at least 50%, or at least 50%, or at least 60%, or at least 70%, or at least 80%, or at least 90%, or at least 1.5 fold, or at least 2 fold, or at least 3 fold, or at least 4-fold, or at least 5-fold higher than said normal control.

6. The method according to any one of claims 1-4, wherein said increased level of kallikrein protease activity comprises a kallikrein activity level at least 5-fold higher than said normal control.

7. The method according to any one of claims 1-6, wherein said substantially equivalent level of kallikrein protease activity in comparison to kallikrein protease activity from a plasma sample of a normal control comprises a statistically insignificant difference from said control at a 5%, or at a 1% confidence level.

8. The method according to any one of claims 1-7, wherein said essentially equivalent level of kallikrein protease activity comprises a kallikrein protease activity level within ±20%, or within ±15%, or within ±10%, or within ±5% of said control level.

9. The method of claim 8, wherein said essentially equivalent level of kallikrein protease activity comprises a kallikrein protease activity level within ±20% of said control level.

10. The method according to any one of claims 1-9, wherein said normal control comprises a level of kallikrein protease activity determined from a population known not to have angioedema.

11. The method according to any one of claims 1-9, wherein said normal control comprises a level of kallikrein protease activity from a subject determined not to have angioedema.

12. The method of claim 11, wherein said level of kallikrein protease activity from a subject determined not to have angioedema comprises a level of kallikrein protease activity determined from said patient at a time when said subject patient is asymptomatic for angioedema.

13. The method of claim 11, wherein said level of kallikrein protease activity from a subject determined not to have angioedema comprises a level of kallikrein protease activity determined from a subject that is not said patient.

14. The method according to any one of claims 1-13, wherein the kallikrein stimulating agent or the activator of the plasma contact system comprises an agent selected from the group consisting of dextran sulfate, or polyphosphates.

15. The method of claim 14, wherein the kallikrein stimulating agent or the activator of the plasma contact system comprises dextran sulfate (DXS).

16. The method of any one of claims 1-15, wherein the plasma sample is subjected to or exposed to kallikrein stimulating agent or the activator of the plasma contact system at one or more concentrations in the range of from about 0.5 μg/mL, or from about 1.0 μg/mL, or from about 1.25 μg/mL up to about 10 μg/mL, or up to about 9 μg/mL, or up to about 8 μg/mL, or up to about 7 μg/mL, or up to about 6 μg/mL, or up to about 5 μg/mL, or up to about 4 μg/mL, or up to about 3 μg/mL, or up to about 2 μg/mL.

17. The method of claim 16, wherein the plasma sample is subjected to or exposed to kallikrein stimulating agent or the activator of the plasma contact system a concentration ranging from about 1 μg/mL up to about 5 μg/mL, or from about 1.25 μg/mL up to about 4 μg/mL, or at from about 1.5 μg/mL up to about 3 μg/mL, or at about 2.0 μg/mL up to about 3.0 μg/mL.

18. The method of any one of claims 1-17, wherein measuring kallikrein protease activity in said sample comprises stimulating said sample with a kallikrein substrate and detecting and/or quantifying cleavage of said substrate.

19. The method of claim 18, wherein the kallikrein substrate comprises Z-Phe-Arg-AMC-HCl.

20. The method according to any one of claims 18-19, wherein cleavage of the kallikrein substrate is detected via a fluorescent, chromogenic, chemiluminescent, radioactive or enzymatic signal.

21. The method of any one of claims 1-20, wherein the plasma sample is subjected or exposed to kallikrein stimulating agent or the activator of the plasma contact system for about 60 minutes or less, or for about 50 minutes or less, or for about 40 minutes or less, or for about 30 minutes or less. or for about 20 minutes or less, or for about 10 minutes or less.

22. The method according to any one of claims 1-21, wherein the kallikrein protease activity level(s) are normalized to rKLKB1.

23. The method according to any one of claims 1-22, wherein said plasma sample comprises a frozen sample that has been thawed once that has been thawed once at 37° C. to be assayed.

24. The method according to any one of claims 1-22, wherein said plasma sample comprises a fresh (unfrozen) plasma sample.

25. The method according to any one of claims 1-22, wherein said plasma sample has been stored as a frozen plasma sample.

26. The method according to any one of claims 1-25, wherein said identifying an identifying an increased level of kallikrein protease activity comprises diagnosing said patient as a patient with a bradykinin-mediated angioedema.

27. The method of claim 26, where said diagnosing is in the context of a differential diagnosis for bradykinin-mediated angioedema.

28. The method according to any one of claims 26-27, wherein said diagnosing comprises identifying said patient as a patient suffering hereditary angioedema due to C1 inhibitor deficiency (HAE-C1INH), hereditary angioedema with normal C1 inhibitor (HAE-nl-CINH) and/or idiopathic non-histaminergic angioedema (INHA).

29. The method according to any one of claims 1-25, wherein said identifying an identifying an essentially equivalent level of kallikrein protease activity comprises diagnosing said patient as a patient with a histamine-mediated angioedema.

30. The method of claim 29, where said diagnosing is in the context of a differential diagnosis for histamine-mediated angioedema.

31. The method according to any one of claims 1-30, wherein said method further comprises recording said level of kallikrein protease activity in a patient medical record.

32. The method according to any one of claims 1-31, wherein said method further comprises recording said angioedema as a bradykinin-mediated angioedema or a histamine-mediated angioedema in a medical record.

33. The method according to any one of claims 1-31, wherein said method further comprises recording in a medical record said subject as a subject with hereditary angioedema due to C1 inhibitor deficiency (HAE-C1INH), hereditary angioedema with normal C1 inhibitor (HAE-nl-CINH) and/or idiopathic non-histaminergic angioedema (INHA).

34. The method according to any one of claims 31-33, wherein the patient medical record is a medical record maintained by a laboratory, physician's office, a hospital, a health maintenance organization, an insurance company, or a personal medical record website.

35. The method according to any one of claims 31-34, wherein information from said medical record is recorded on or in a medic alert article selected from a card, worn article, or radiofrequency identification (RFID) tag.

36. A method of treating a patient for angioedema, said method comprising:
using a plasma sample from said patient, performing a method according to any one of claims 1-35 to identify said subject as having a bradykinin-mediated angioedema or a histamine-mediated angioedema; and
where said subject is identified as having a bradykinin-mediated angioedema, treating said subject for bradykinin-mediated angioedema; and
where said subject is identified as having a histamine-mediated angioedema, treating said subject for histamine-mediated angioedema.

37. A method of treating a subject for angioedema, said method comprising:
in a subject identified by a method according to any one of claims 1-35 as having a bradykinin-mediated angioedema, treating said subject for bradykinin-mediated angioedema; or
in a subject identified by a method according to any one of claims 1-35 as having a histamine-mediated angioedema, treating said subject for histamine-mediated angioedema.

38. The method according to any one of claims 36-37, wherein said treating said subject for bradykinin-mediated angioedema comprises administering to, or causing to be administered to, said subject a bradykinin-targeted medication.

39. The method of claim 38, wherein said bradykinin-targeted medication comprises one or more agents selected from the group consisting of IV fresh frozen plasma (FFP), epsilon-aminocaproic acid, C1-INH replacement therapy, a plasma kallikrein inhibitor, a C1 esterase inhibitor, and a bradykinin receptor blocker.

40. The method of claim 39, wherein said bradykinin-targeted medication comprises C1-INH (Berinert).

41. The method according to any one of claims 39-40, wherein said bradykinin-targeted medication comprises the plasma kallikrein inhibitor ecallantide or ladalenumab (Takhzyro®) (a humanized monoclonal antibody against Kallikrein).

42. The method according to any one of claims 39-41, wherein said bradykinin-targeted medication comprises the bradykinin receptor blocker icatiban.

43. The method according to any one of claims 39-42, wherein said bradykinin-targeted medication comprises the C1 esterase inhibitor, plasma sources (Berinert,® Haegarda®, Cynrize® or recombinant ruconest.

44. The method according to any one of claims 36-37, wherein said treating said subject for histamine-mediated angioedema comprises administering to, or causing to be administered to, said subject a histamine-targeted medication.

45. The method of claim 38, wherein said histamine targeted medication comprises one or more agents selected from the group consisting of H1 receptor antagonists and drugs that are not histamine targeted but that are needed due to the adrenergic pathways and inflammatory response that become activated by histamine release (e.g., epinephrine, an antihistamine, a β2 agonist, a corticosteroid.

46. The method of claim 45, wherein said histamine targeted medication comprises epinephrine.

47. The method according to any one of claims 45-46, wherein said histamine targeted medication comprises a corticosteroid.

48. The method of claim 47, wherein said corticosteroid comprises methylprednisolone.

49. The method according to any one of claims 45-48, wherein said histamine targeted medication comprises an antihistamine.

50. The method according to any one of claims 45-49, wherein said B2 receptors targeted medication comprises a β2 agonist.

51. The method of claim 50, wherein said β2 agonist comprises albuterol.

52. The method according to any one of claims 45-51, wherein said histamine-targeted medication comprises a mast-cell targeted medication comprising a leukotriene modifier or omalizumab.

53. A method of identifying a patient suffering bradykinin-mediated angioedema, comprising:
a) subjecting or exposing a plasma sample from the patient to a kallikrein stimulating agent or an activator of the plasma contact system in an amount sufficient to stimulate kallikrein protease activity;
b) measuring the kallikrein protease activity; and
c) identifying an increased level of kallikrein protease activity in comparison to kallikrein protease activity from a plasma sample of a normal control as indicative that the patient is suffering bradykinin-mediated angioedema.

54. The method of claim 53, further comprising administering a bradykinin-targeted medication to the patient.

55. The method of any one of claims 53 to 54, wherein the kallikrein stimulating agent or the activator of the plasma contact system comprises dextran sulfate (DXS).

56. The method of any one of claims 53 to 55, wherein the plasma sample is subjected or exposed to kallikrein stimulating agent or the activator of the plasma contact system at one or more concentrations in the range of from about 1 μg/mL to about 10 μg/mL, e.g., 1 μg/mL, 1.25 μg/mL, 2 μg/mL 2.5 μg/mL 3 μg/mL 4 μg/mL 5 μg/mL 6 μg/mL 7 μg/mL 8 μg/mL 9 μg/mL or 10 μg/mL.

57. The method of any one of claims 53 to 56, wherein the plasma subjected or exposed to the kallikrein stimulating agent or the activator of the plasma contact system is contacted with a kallikrein substrate.

58. The method of claim 57, wherein the kallikrein substrate comprises Z-Phe-Arg-AMC-HCl.

59. The method of claim 57, wherein cleavage of the kallikrein substrate is detected via a fluorescent, chromogenic, chemiluminescent, radioactive or enzymatic signal.

60. The method of any one of claims 53 to 59, wherein the plasma sample is subjected or exposed to kallikrein stimulating agent or the activator of the plasma contact system for 60 minutes or less, e.g., 50 minutes, 40 minutes, 30 minutes, or less.

61. The method of any one of claims 53 to 60, wherein the method identifies patients suffering hereditary angioedema due to C1 inhibitor deficiency (HAE-C1INH), hereditary angioedema with normal C1 inhibitor (HAE-nl-CINH) and/or idiopathic non-histaminergic angioedema (INHA).

62. A method of identifying a patient suffering histamine-mediated angioedema, comprising:
a) subjecting or exposing a plasma sample from the patient to a kallikrein stimulating agent or an activator of the plasma contact system in an amount sufficient to stimulate kallikrein protease activity;
b) measuring the kallikrein protease activity; and
c) identifying an essentially equivalent level of kallikrein protease activity in comparison to kallikrein protease activity from a plasma sample of a normal control as indicative that the patient is suffering histamine-mediated angioedema.

63. The method of claim 62, further comprising administering a histamine-targeted medication to the patient.

64. The method of any one of claims 62 to 63, wherein the kallikrein stimulating agent or the activator of the plasma contact system comprises dextran sulfate (DXS).

65. The method of any one of claims 62 to 64, wherein the plasma sample is subjected or exposed to kallikrein stimulating agent or the activator of the plasma contact system at one or more concentrations in the range of from about 1 μg/mL to about 10 μg/mL, e.g., 1 μg/mL, 1.25 μg/mL, 2 μg/mL 2.5 μg/mL 3 μg/mL 4 μg/mL 5 μg/mL, 6 μg/mL, 7 μg/mL 8 μg/mL 9 μg/mL, or 10 μg/mL.

66. The method of any one of claims 62 to 65, wherein the plasma subjected or exposed to the kallikrein stimulating agent or the activator of the plasma contact system is contacted with a kallikrein substrate.

67. The method of claim 66, wherein the kallikrein substrate comprises Z-Phe-Arg-AMC-HCl.

68. The method of claim 66, wherein cleavage of the kallikrein substrate is detected via a fluorescent, chromogenic, chemiluminescent, radioactive or enzymatic signal.

69. The method of any one of claims 62 to 68, wherein the plasma sample is subjected or exposed to kallikrein stimulating agent or the activator of the plasma contact system for 60 minutes or less, e.g., 50 minutes, 40 minutes, 30 minutes, or less.
